(19) United States
(12) Patent Application Publication
(10) Pub. No.: US 2018/0079997 A1
(43) Pub. Date: Mar. 22, 2018

(54) IMPROVED BIOGAS PRODUCTION SYSTEM AND METHOD OF MANUFACTURE THEREOF

(71) Applicant: KRU Energy Asia Pte Ltd, Singapore (SG)

(72) Inventor: JT KRUEGER, Singapore (SG)

(73) Assignee: KRU Energy Asia Pte Ltd, Singapore (SG)

(21) Appl. No.: 15/566,662

(22) PCT Filed: Apr. 18, 2016
(86) PCT No.: PCT/SG2016/050181

§ 371 Date: Oct. 13, 2017

(30) Foreign Application Priority Data
Apr. 16, 2015 (SG) 10201503024X

(51) Int. Cl. C12M 1/107; C12M 1/00; C12M 1/02

(52) U.S. Cl. CPC C12M 21/04; C12M 23/58; C07C 9/04; C12M 29/06; C12M 41/18; C12M 29/02

(57) ABSTRACT

The present invention provides a biogas production system comprising: at least one digester for generating biogas and digestate, each digester comprising: a first end portion, a second end portion, and a peripheral side-wall there-between; one or more inlets, wherein at least one inlet is arranged to receive organic substrates; one or more outlets, wherein at least one outlet is arranged to release biogas; and a feed system comprising a pit arranged to receive and supply said organic substrates to the digester, said inlet positioned adjacent to a lower portion of said pit, the pit arranged such that a weight of the organic substrates within the pit bias the organic substrates into the digester.

### FIELD OF THE INVENTION

The invention relates to the production of biogas, and in particular to a system and method of manufacturing biogas.

### BACKGROUND

Conventionally, electricity is generated by burning fuel such as coal and diesel. These methods deplete the world's natural resources and generate greenhouse gases, which have an adverse impact on the environmental.

The generation of clean and combustible biogas (mainly methane and carbon dioxide) from anaerobic biomass digestion is becoming an increasingly popular method for electricity generation due to the many benefits associate with them. Importantly, the production of biogas is considered non-polluting in nature in that it uses up waste materials found in landfills, dumpsites and farms, and so allow for a decreased in soil and water pollution. Further, the production of biogas does not require combustible fuel and oxygen, which means that resources are conserved by not using any further fuel and natural resources.

However, operating and maintaining of existing biogas production systems come with its challenges. An existing biogas production system uses motor driven augers and/or force feeding machines for providing a regular supply of organic substrates to the digester. To this end, the operation and maintenance of the said augers and/or feeding machines, in addition to equipment for transporting the organic substrates from a discrete location (landfills, dumpsites and farms, for example) to the biogas production facility, require extensive as well as additional energy loads and manpower, which in turn drives up the capital and operating expenditure.

Another system uses mechanically driven in-digester and/or on-digester mixers, such as a paddle mixer for example, and in-digester and/or on-digester heating elements for churning, and heating the organic substrate during anaerobic digestion. This arrangement makes regular maintenance, repair and/or replacement of the said mixers and heating elements necessary in order to ensure effective and smooth anaerobic digestion. In other words, draining of the digester and disruption to the biogas production would be inevitable in order to make way for regular maintenance, repair and/or replacement activities. In addition, the use of moving mechanical parts also leads to reliability issues.

To this end, existing biogas production systems have limited industrial deployment due to issues on cost, design and operation. In particular, it remains a challenge to design a biogas production system that is reliable, energy and cost efficient as well as easy to operate and maintain.

### SUMMARY OF INVENTION

In a first aspect the invention provides a biogas production system comprising: at least one digester for generating biogas and digestate, each digester comprising: a first end portion, a second end portion, and a peripheral side-wall there-between; one or more inlets, wherein at least one inlet is arranged to receive organic substrates; one or more outlets, wherein at least one outlet is arranged to release biogas; and a feed system comprising a pit arranged to receive and supply said organic substrates to the digester, said inlet positioned adjacent to a lower portion of said pit, the pit arranged such that a weight of the organic substrates within the pit bias the organic substrates into the digester.

In an embodiment of the present invention, the biogas production system may further comprise at least one discrete mixing unit, said at least one mixing unit arranged to withdraw a portion of the organic substrates from the digester, and to reintroduce said portion of organic substrates to a surface of the organic substrate.

The biogas production system may further comprise at least one discrete heating unit arranged to heat the portion of organic substrates.

In a particular embodiment of the present invention, the heating unit may comprise one or more heat exchangers.

In a further embodiment of the present invention, the heating unit may be arranged to heat the portion of organic substrates in the temperature range of 40° C. to 42° C.

In yet a further embodiment, the mixing unit may comprise at least one swivelling spray nozzle positioned above the inlet for receiving the organic substrates.

In one embodiment of the present invention, the at least one mixing unit and at least one heating unit may be provided in a portable containerised unit.

In a particular embodiment of the present invention, the feed system may comprise an inclined platform proximate to the first end portion of the digester, said platform arranged to define one or more flow paths between the inlet for receiving organic substrates and the digester for supplying said organic substrates under gravity.

In a further embodiment of the present invention, the biogas production system may further comprise at least one digestate processing unit arranged to recover nutrients from the digestate so as to generate fertilizers.

In yet a further embodiment, the recovered nutrients may comprise one or a combination of: nitrogen, potassium and phosphorus.

In a particular embodiment of the present invention, the digestate processing unit may comprise one or more micro, ultra and/or nano-filters.

In an embodiment of the present invention, the digestate processing unit may be connected to one or more third party locations and/or a storage facility, said biogas production system further comprising a shutoff system arranged to cut off a supply of fertilizers to non-paying third parties.

In a second aspect the invention provides a method of biogas production comprising the steps of: providing at least one digester for generating biogas and digestate, each digester comprising: a first end portion, a second end portion, and a peripheral side-wall there-between; one or more inlets, wherein at least one inlet is arranged to receive organic substrates; one or more outlets, wherein at least one outlet is arranged to release biogas; and providing a feed system for feeding organic substrate to the at least one digester, the feed system comprising a pit arranged to receive and supply said organic substrates to the digester, said inlet positioned adjacent to a lower portion of said pit, the pit arranged such that a weight of the organic substrates within the pit bias the organic substrates into the digester.

In an embodiment of the present invention, the method may further comprise the steps of: withdrawing a portion of the organic substrate from the digester following the feeding step; and reintroducing said portion of organic substrates to a surface of the organic substrate.

In one embodiment of the present invention, the method may further comprise the step of heating the portion of organic substrates prior to the reintroduction step.

In a further embodiment of the present invention, the portion of organic substrate may be reintroduced at a level above the inlet for receiving organic substrates.

In yet a further embodiment of the present invention, the method may further comprise the step of processing the digestate to generate fertilizers.

In a particular embodiment of the present invention, the step of processing the digestate may involve micro, ultra and/or nano-filtration of said digestate.

In a third aspect the invention provides a discrete mixing unit for use with a digester comprising organic substrates, said mixing unit arranged to withdraw a portion of the organic substrates from a base portion of the digester, and to reintroduce said portion of organic substrates to a surface of the organic substrate.

In one embodiment of the present invention, the mixing unit may comprise at least one discrete heating unit arranged to heat the portion of organic substrates.

### BRIEF DESCRIPTION OF DRAWINGS

It will be convenient to further describe the present invention with respect to the accompanying drawings that illustrate possible arrangements of the invention. Other arrangements of the invention are possible and consequently, the particularity of the accompanying drawings is not to be understood as superseding the generality of the preceding description of the invention.

FIG. 1 is a schematic drawing of a biogas production system according to one embodiment of the present invention; and

FIG. 2 is a side-elevation blown-up view of a feed system according to one embodiment of the present invention;

FIG. 3 is a flow chart showing a method of biogas production according to one embodiment of the present invention.

### DETAILED DESCRIPTION

Organic substrates (pure mono-substrates or a mixture of various organic substrates, along with the presence of water) are decomposed in an anaerobic reactor or digester (air-tight) by a variety of micro-organisms (fermentative bacteria, acetogenic bacteria and methanogenic bacteria) present in the organic substrate through a complex biological process in the absence of oxygen, known as anaerobic fermentation or anaerobic digestion. As a result of anaerobic digestion, biogas and digestate are formed.

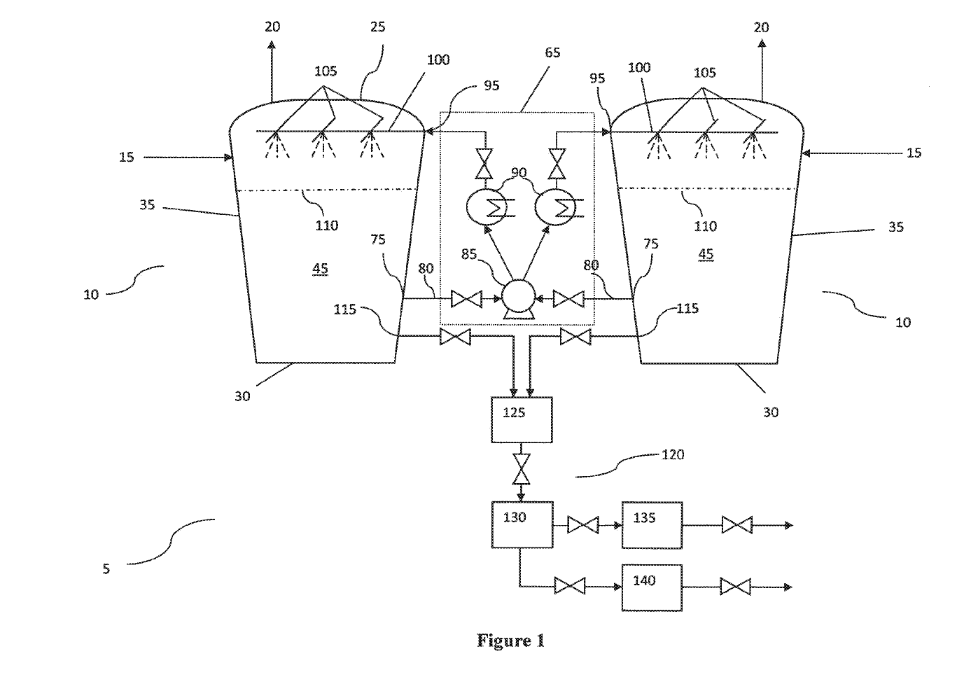

FIG. 1 shows a schematic diagram of a biogas production system according to one embodiment of the present invention. In this embodiment, the biogas production system 5 comprises two digesters 10. Each digester may comprise a first end portion 25, a second end portion 30 and a peripheral side-wall 35 there between. The digester 10 may comprise one or more inlets and one or more outlets. At least one inlet 15 may be arranged to receive organic substrates 45, which may include, but are not limited to, energy corps (cereals, corn, millet, for example), liquid or solid manure (cattle and poultry manure for example), domestic organic wastes (fruit and vegetable wastes for example), or a combination thereof. At least one outlet 20 may be arranged to release biogas. It will be appreciated that the outlets 20 may be connected to one or more cogeneration units (also known as combined heat and power units) in order to produce electricity and useful heat.

It will be appreciated that the number of digesters in the biogas production system may vary according to site limitations (land size for example) and the intended application (amount and type of organic substrates being processed as well as the desired biogas production capacity, for example).

In any embodiment of the present invention, the digester may be constructed using any suitable material that is inert to the reaction environment in the digester, and resistant to corrosion. For example, suitable materials may include but are not limited to a bricks, concrete, rebars or a combination thereof.

In one embodiment of the present invention, the first end portion 25 of the digester 10 may be a dome shaped cover or roof. As a result of anaerobic digestion, biogas is formed in the digester, which starts to collect in the dome shaped roof. In particular, a digester with a dome shaped roof can contain a larger volume of biogas for a given surface area as compared to one with a flat roof, and so minimize the amount of materials and hence cost required for constructing the digester. In this way, a digester with a dome shaped roof may serve as a more cost efficient pressure containment device for biogas.

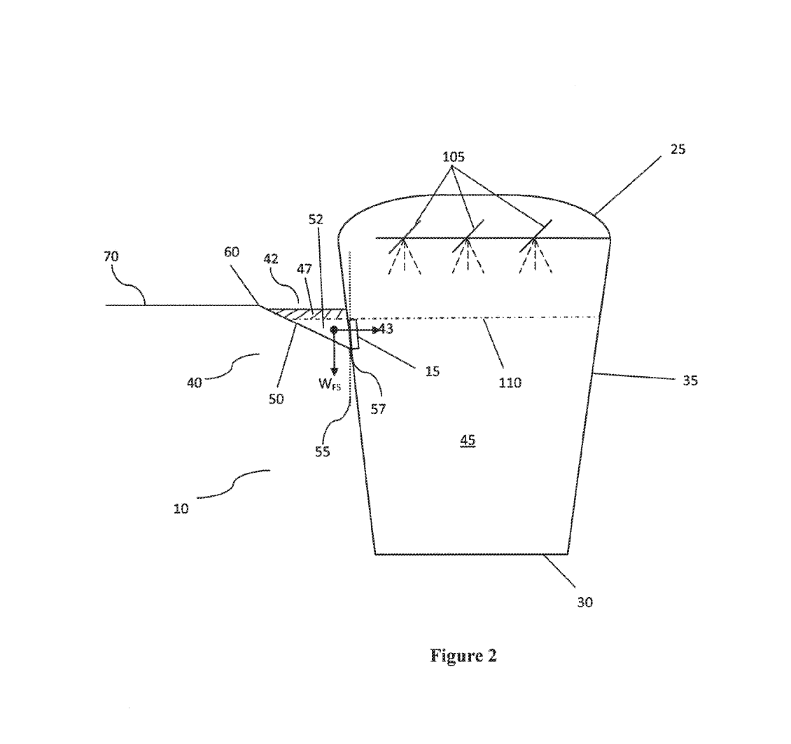

FIG. 2 depicts a feed system arranged to supply organic substrates or feedstock to the digester according to an embodiment of the present invention. The feed system 40 may comprise a recess or pit 42 proximate to the first end portion of the digester 25, arranged to supply organic substrates to the digester 10. The feed system 40 may be configured such that the inlet 15 is at a lower portion 57 of the pit 42. This arrangement advantageously creates a self-feeding system whereby feedstock at the bottom 52 of the pit 42 flows, or is biased 43 into the digester 10 under its own weight W_{FS}. This may occur when a portion of the organic substrates 45 and/or digestate is withdrawn from the digester 10. Excess feedstock 47, that being the pile of feedstock that was stacked above the feedstock at the bottom 52 of the pit 42, is then biased under its own weight to form the next batch of feedstock at the bottom of the pit.

Any suitable method or machinery may be used to transport the supply of organic substrates or feedstock from a neighboring or discrete location (landfills, dumpsites and farms, for example) to the feed system 40. Suitable machineries include, but are not limited to front end loaders or trackers.

In one embodiment of the present invention, the feed system 40 may comprise an inclined platform 50 proximate to the first end portion 25 of the digester 10. By inclining the platform 50 to the vertical 55, the organic substrates or feedstock may travel through the platform 50 from the leading edge 60 of the platform 50 to the digester 10 via inlet 15 with the help of gravity. In this way, one or more flow paths between the inlet 15 and digester 10 may be defined for supplying organic substrates 45 under gravity.

To this end, the feed system 40 of the present invention advantageously reduces the work, energy and costs involved in force feeding the digesters.

The platform 50 may be formed using any suitable material that is inert to the reaction environment in the digester, and resistant to corrosion. For example, suitable materials may include but are not limited to bricks, metal (stainless steel for example), concrete or a combination thereof.

Additionally, the digester in the embodiment depicted in FIG. 1 may comprise a frusto-conical peripheral side-wall 35. Using the force of gravity, the organic substrates or feedstock 45 introduced by the feed system 40 may be packed and compacted within the digester 10. Preferably, the digester 10 may be underground and arranged such that the inlet 15 is just above the trailing end 57 of the platform 50. It will be appreciated that an above ground digester may also be used along with a feed system provided on an elevated platform, and so adapted to be suitable for supplying organic substrates under gravity.

Accordingly, the feed system 40 of the present invention has an advantage over conventional biogas production systems on operation, maintenance and repair by using less machinery and equipment for the supply of organic substrates to the digester. The digester 10 depicted in FIG. 1 may be provided with at least one discrete or ex-digester mixing unit 65 for mixing and/or churning of the organic substrate 45 within digester 10. In particular, the mixing unit 65 ensures contact between the active biomass, that is organic substrates undergoing anaerobic digestion, and the newly introduced organic substrates or feedstock so as to allow the micro-organisms to work more quickly, and aid in the digestion process. The mixing unit 65 also serves to ensure temperature uniformity between the active biomass and feedstock, thereby preventing the formation of a temperature gradient. Further, the mixing unit 65 may facilitate the accumulation of biogas generated as a result of anaerobic digestion above the organic substrates.

The digester 10 may be provided with an outlet 75 proximate to the second end portion 30 for connection to a pipeline 80 of the mixing unit 65 depicted in FIG. 1. The mixing unit 65 may include a pump 85 downstream of said outlet 75 for withdrawing organic substrates or active biomass from the bottom or base of the digester 10. It will be appreciated that any suitable pumps may be used. For example, suitable pumps may include, but are not limited to, centrifugal pump, positive displacement pump or a combination thereof. It will be appreciated that the type pump used may vary according to the type of organic substrates being processed. For instance, positive displacement pump would be more suitable for substrates with higher solids content.

Additionally, the mixing unit 65 may be provided with a discrete or ex-digester heating unit 90 downstream of said pump 85. The heating unit 90 is arranged to heat the withdrawn organic substrates or active biomass, prior to returning the said organic substrates back into digester 10, at a pre-determined temperature to enable the microbial activity necessary for biogas production. Any suitable heating element may be used. Suitable heating elements may include, but are not limited to, ex-digester/reactor heat exchangers. In countries with higher temperatures and longer sunshine hours, solar-heated water may be a cost-effective solution for heating. Preferably, the heating unit 90 is arranged to heat the withdrawn organic substrate or active biomass in the temperature range of 40° C. to 42° C.

Further, the digester 10 may be provided with an inlet 95 for connection to a pipeline 100 and a plurality of outlets 105 of the mixing unit 65. The pipeline 100 and outlets 105 serve to reintroduce the said heated organic substrate or active biomass to a surface 110 of the newly fed organic substrate or feedstock. The plurality of outlets 105 may include one or more swivelling spray nozzles positioned above inlet 15. Preferably, the one or more spray nozzles turn at a 90 degree angle gradually over a period of 24 hours.

Further still, the digester 10 according to any one embodiment of the present invention may be provided with an insulating layer (by covering the digester with earth for example) so as to be well suited for biogas production systems in temperate areas.

Whilst the afore-mentioned arrangement of pump and heating units have been identified for the mixing unit 65 depicted in FIG. 1, the skilled person will appreciate that other arrangements may be used. Alternative arrangements comprising one or more back-up pumps and one or more heating elements may be used, subject to the operating conditions and process parameters of the biogas production system (desired running time, amount of substrates passing through the mixing unit, heating and/or withdrawing capacity of the mixing unit, for example). In particular, an alternative arrangement may comprise an additional back-up pump so that when one pump fails, this dual pump arrangement may allow a switch from the faulty pump to the back-up pump, and so avoids disrupting the biogas production process.

In an embodiment of the present invention, the mixing unit 65 and heating units 90 may be provided in a portable containerised unit to permit easy and quick installation of said mixing and heating units to the digesters of the biogas production system, as well as easy transportation between biogas project sites.

Accordingly, the operation of the afore-mentioned ex-digester mixing 65 and heating 90 units serves to pile active biomass immediately above the surface of the newly introduced feedstock. In this way, a natural air tight barrier is advantageously created between the external environment and the inlet 15 of the digester 10. This prevents oxygen from entering the digester and so creates a conducive environment for anaerobic digestion.

An additional advantage with respect to the mixing and heating units of the present invention is the reduction of in-digester motor and mechanical equipment. By having ex-digester mixing units and heating elements, maintenance and replacement activities for the claimed biogas production system are much easier as compared to conventional biogas production systems having in-digester/on-digester mixers and/or heating elements. In particular, the claimed biogas production system allows for easy installation, maintenance and repair whilst reducing downtime for biogas production.

In the embodiment of FIG. 1, digested organic substrate or digestate is removed via an outlet 115 provided in the digester 10. The digestate may undergo further processing in order to meet local environmental regulations, whereby nutrients and organic matter are required to be removed before discharge into a receiving body and/or limitations in nutrient loading on farmlands. Further, it will be appreciated that the digestate may be diluted with respect to its content of plant nutrients (nitrogen, phosphorus, potassium and sulphur for example). This results in significant costs in the handling, transport and storage of large volume of digestate with low nutritional concentration.

The biogas production system 5 of FIG. 1 may be provided with a digestate processing system 120 comprising one or more digestate processing unit 130, subject to the content of the organic substrate feedstock and digestate. To this end, the digestate processing system 120 of the present invention serves to:
recover nutrients from the digestate so as to generate end-products (fertilisers and/or soil conditioners for agriculture, horticulture, forestry) with higher concentrations of plant nutrients than the unprocessed digestate, and to reduce handling, transportation and storage costs; and
condition said digestate in order to meet local environmental regulations prior to discharge to receiving waters and/or use on farmlands, for example.

The digestate processing system 120 may include one or more holding tanks 125 upstream of the digestate processing unit 130 whereby the digestate generated from the digesters 10 may be stored, and processed according to supply and demand.

In any one embodiment of the present invention, the digestate processing unit 130 may utilise any suitable process and/or equipment for achieving the desired level of nutrient recovery and digestate conditioning. Suitable process and/or equipment include, but are not limited to, decanter centrifuges, discontinuous centrifuges, belt filter presses, vacuum presses, screw press separators, flotation, drying, evaporation, vibrating screen separators, or a combination thereof.

In one embodiment of the present invention, the digestate processing unit 130 may comprise one or more micro, ultra, and/or nano-filters (membrane purification technology) for producing a nutrient concentrate or fertilizer, and purified water. Subject to the content of the digestate, membrane purification technology may be used along with one or a combination of the afore-mentioned digestate processing methods and/or equipment for achieving the desired level of nutrient recovery and digestate conditioning.

Among the technologies for further treatment/processing of the digestate, membrane purification is the only process that can achieve a degree of purification that can allow direct discharge of the purified water to receiving waters. Further, the relatively simple process of membrane purification has the additional advantage of producing nutrient concentrates with nitrogen, phosphorous and potassium levels unmatched in the industry.

The digestate processing system 120 according to the embodiment in FIG. 1 may additionally comprise one or more storage tanks 135 downstream of the digestate processing unit 130. These storage tanks 135 serve to accommodate the processed nutrient concentrate or fertilisers. The storage tanks 135 may be connected to one or more third party locations (agriculture or horticulture farmlands for example) for supply of said nutrient concentrate or fertilisers.

In any one embodiment of the present invention, the biogas production system 10 and digestate processing system 120 may comprise a shutoff system arranged to cut off a supply of fertilizers to non-paying third parties.

Further, the digestate processing system 120 may be provided with one or more storage tanks 140 downstream of the digestate processing unit 130 for accommodating the purified water. The storage tanks 140 may be connected to one or more receiving bodies via drains and sewage pipes.

In any one embodiment of the present invention, the biogas production system 10 and digestate processing system 120 may be provided with the required power supply, under conditions known to a person skilled in the art, for proper functioning of the operating units.

In a specific embodiment of the present invention, the pipelines of the biogas production system 10 and digestate processing system 120 may be stainless steel pipelines, so as to protect said pipelines against corrosive elements and/or chemicals in the digester.

In any embodiment of the present invention, the biogas production system 10 and digestate processing system 120 may be provided with one or more valves for safety (shutoff and isolating elements to reduce and/or avoid danger, or contamination), and to facilitate maintenance and repair. Any suitable valves may be used. For example, suitable valves may include, but are not limited to, ball valves and/or cock valves along with T-joints.

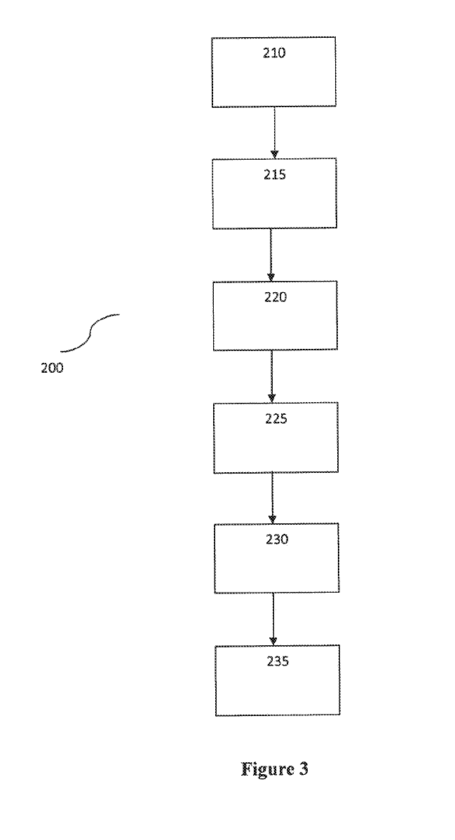

FIG. 3 shows a flow chart of a method of biogas production according to one embodiment of the present invention. Here, the method 200 commences with providing at least one digester for generating biogas and digestate 210. The digester may be a conventional digester or a digester according to any embodiment of the present invention. Next, the digester is being fed 215 with organic substrates or feedstock under gravity using a feed system. The feed system may be a conventional feedstock distribution system, or a feed system according to any embodiment of the present invention.

Following the feeding step 215, the organic substrates or feedstock are being withdrawn 220 from the digester, and reintroduced 230 to a surface of the newly introduced organic substrates or feedstock within the digester to facilitate mixing and/or churning of the organic substrates. Prior to the reintroduction step 230, the withdrawn organic substrates are heated 225. Preferably, the heated organic substrates are reintroduced back to the digester via an inlet positioned at a level above the inlet for receiving the organic substrates or feedstock. This arrangement allows the piling of active biomass immediately above the surface of the newly introduced feedstock. In this way, a natural air tight barrier is advantageously created between the external environment and the digester. This prevents oxygen from entering the digester and so creates a conducive environment for anaerobic digestion.

The digestate generated from the digester may undergo further processing 235, via a digestate processing system, in order to generate fertilizers or nutrient concentrate. The digestate processing system may comprise any one or a combination of conventional treatment methods (decanter centrifuges, belt filter presses, vacuum presses, screw press separators, flotation, drying, for example), or may be a digestate processing system according to any embodiment of the present invention. Preferably, the digestate processing system may comprise the method of micro, ultra and/or nano-filtration in order to generate a nutrient concentrate or fertilizer, and purified water.

### Example 1

32.8 tonnes (weighted average) of organic substrate comprising a mixture of cattle manure (25% dry mass), sorghum silage (28% dry mass), crushed and grounded cereals and grains (87% dry mass) was introduced into a digester, which is designed according to biogas production system of FIG. 1, along with water. The organic substrates contain micro-organisms for anaerobic digestion.

The methane content of the biogas produced was around 52.3 tonnes (weighted average). The biogas production process was operated at a temperature range of 40° C.-42° C.

Based on the above results, the biogas production system according to the embodiment of FIG. 1 yielded sufficient energy for cogeneration plants with 2462 kW of electric power.

### Example 2

29.5 tonnes (weighted average) of organic substrate comprising a mixture of cattle manure (25% dry mass), sorghum silage (28% dry mass), poultry manure (40% dry mass), crushed and grounded cereals and grains (87% dry mass) was introduced into a digester, which is designed according to biogas production system of FIG. 1, along with water. The organic substrates contain micro-organisms for anaerobic digestion.

The methane content of the biogas produced was around 52.6 tonnes (weighted average). The biogas production process was operated at a temperature range of 40° C.-42° C.

Based on the above results, the biogas production system according to the embodiment of FIG. 1 yielded sufficient energy for cogeneration plants with 2245 kW of electric power.

1. A biogas production system comprising:
at least one digester for generating biogas and digestate, each digester comprising:
a first end portion, a second end portion, and a peripheral side-wall there-between;
one or more inlets, wherein at least one inlet is arranged to receive organic substrates;
one or more outlets, wherein at least one outlet is arranged to release biogas; and
a feed system comprising a pit arranged to receive and supply said organic substrates to the digester, said inlet positioned adjacent to a lower portion of said pit, the pit arranged such that a weight of the organic substrates within the pit bias the organic substrates into the digester.

2. The biogas production system according to claim 1, further comprising at least one discrete mixing unit, said at least one mixing unit arranged to withdraw a portion of the organic substrates from the digester, and to reintroduce said portion of organic substrates to a surface of the organic substrate.

3. The biogas production system according to claim 2, further comprising at least one discrete heating unit arranged to heat the portion of organic substrates.

4. The biogas production system according to claim 3, wherein the heating unit comprises one or more heat exchangers.

5. The biogas production system according to claim 3, wherein the heating unit is arranged to heat the portion of organic substrates in the temperature range of 40° C. to 42° C.

6. The biogas production system according to claim 2, wherein the mixing unit comprises at least one swivelling spray nozzle positioned above the inlet for receiving the organic substrates.

7. The biogas production system according to claim 2, wherein the at least one mixing unit and at least one heating unit are provided in a portable containerised unit.

8. The biogas production system according to claim 1, wherein said feed system comprises an inclined platform proximate to the first end portion of the digester, said platform arranged to define one or more flow paths between the inlet for receiving organic substrates and the digester for supplying said organic substrates under gravity.

9. The biogas production system according to claim 1, further comprising at least one digestate processing unit arranged to recover nutrients from the digestate so as to generate fertilizers.

10. The biogas production system according to claim 9, wherein the recovered nutrients comprises one or a combination of: nitrogen, potassium and phosphorus.

11. The biogas production system according to claim 9, wherein the digestate processing unit comprises one or more micro, ultra and/or nano-filters.

12. The biogas production system according to claim 9, wherein the digestate processing unit is connected to one or more third party locations and/or a storage facility, said biogas production system further comprising a shutoff system arranged to cut off a supply of fertilizers to non-paying third parties.

13. A method of biogas production comprising the steps of:
providing at least one digester for generating biogas and digestate, each digester comprising:
a first end portion, a second end portion, and a peripheral side-wall there-between;
one or more inlets, wherein at least one inlet is arranged to receive organic substrates;
one or more outlets, wherein at least one outlet is arranged to release biogas; and
providing a feed system for feeding organic substrate to the at least one digester, the feed system comprising a pit arranged to receive and supply said organic substrates to the digester, said inlet positioned adjacent to a lower portion of said pit, the pit arranged such that a weight of the organic substrates within the pit bias the organic substrates into the digester.

14. The method of biogas production according to claim 13, further comprising the steps of:
withdrawing a portion of the organic substrate from the digester following the feeding step; and
reintroducing said portion of organic substrates to a surface of the organic substrate.

15. The method of biogas production according to claim 14, further comprising the step of heating the portion of organic substrates prior to the reintroduction step.

16. The method of biogas production according to claim 14, wherein the portion of organic substrate is reintroduced at a level above the inlet for receiving organic substrates.

17. The method of biogas production according to claim 15, further comprising the step of processing the digestate to generate fertilizers.

18. The method of biogas production according to claim 17, wherein the step of processing the digestate involves micro, ultra and/or nano-filtration of said digestate.

19. A discrete mixing unit for use with a digester comprising organic substrates, said mixing unit arranged to withdraw a portion of the organic substrates from a base portion of the digester, and to reintroduce said portion of organic substrates to a surface of the organic substrate.

20. The mixing unit according to claim 19, further comprising at least one discrete heating unit arranged to heat the portion of organic substrates.
